(19) United States
(12) Patent Application Publication
(10) Pub. No.: US 2025/0384555 A1
(43) Pub. Date: Dec. 18, 2025

(54) SYSTEM AND METHOD FOR SELECTION OF REFERENCE IMAGES FOR USE IN DETECTING AND TRACKING ARRIVAL OF CONTRAST BOLUS IN CONTRAST-ENHANCED MAGNETIC RESONANCE IMAGING

(71) Applicant: GE Precision Healthcare LLC, Waukesha, WI (US)

(72) Inventors: Vishal Manwani, Bangalore (IN); Deepthi Sundaran, Bangalore (IN); Neha Singh, Bangalore (IN); Jignesh Dholakia, Bangalore (IN); Vignesh Singh, Bangalore (IN)

(21) Appl. No.: 18/744,970

(22) Filed: Jun. 17, 2024

(51) Int. Cl. G06T 7/00; A61B 5/055

(52) U.S. Cl. CPC G06T 7/0014; A61B 5/055; G06T 2200/24; G06T 2207/10088

(57) ABSTRACT

For a given MR image having an anatomical landmark and a frame of reference, a method includes automatically selecting one or more series of MR images from a plurality of series of MR images previously acquired and having the anatomical landmark and the frame of reference. The method includes automatically selecting one or more candidate groups from the one or more series of MR images that were selected, wherein selection of the one or more candidate groups is based on predetermined rules. The method includes automatically selecting a respective reference image from each candidate group, wherein selection of each respective reference image is based on the predetermined rules. The method further includes automatically displaying the respective reference image selected for each candidate group, wherein each respective reference image is displayed in reference viewports on a user interface on a display based on the predetermined rules.

### BACKGROUND

The subject matter disclosed herein relates to medical imaging and, more particularly, to a system and method for selection of reference images for use in detecting and tracking arrival of contrast bolus in contrast-enhanced magnetic resonance imaging.

Magnetic resonance imaging (MRI) is a medical imaging modality that can create images of the inside of a human body without using x-rays or other ionizing radiation. An MRI scan typically includes a series of radiofrequency (RF) excitation pulses and magnetic field gradient pulses that are played out with specific timings and in a specific sequence to prepare contrast and encode spatial information into the signal to generate an image. To enhance certain anatomical features, some MRI scans may include the administration of a contrast agent to a subject being imaged.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for selecting reference images for a magnetic resonance (MR) scan is provided. For a given MR image acquired with an MR scanner and having an anatomical landmark and a frame of reference, the computer-implemented method includes automatically selecting, via a processing system comprising one or more processors, one or more series of MR images from a plurality of series of MR images previously acquired with the MR scanner and having the anatomical landmark and the frame of reference. The computer-implemented method also includes automatically selecting, via the processing system, one or more candidate groups from the one or more series of MR images that were selected, wherein selection of the one or more candidate groups is based on predetermined rules. The computer-implemented method further includes automatically selecting, via the processing system, a respective reference image from each candidate group of the one or more candidate groups that were selected, wherein selection of each respective reference image is based on the predetermined rules. The computer-implemented method still further includes automatically displaying, via the processing system, the respective reference image selected for each candidate group of the one or more candidate groups that were selected, wherein each respective reference image is displayed in reference viewports on a user interface on a display based on the predetermined rules.

In another embodiment, a system for selecting reference images for a magnetic resonance (MR) scan is provided. The system includes a memory encoding processor-executable routines. The system also includes a processing system including one or more processors and configured to access the memory and to execute the processor-executable routines, wherein the processor-executable routines, when executed by the processing system, cause the processing system to perform actions. For a given MR image acquired with an MR scanner and having an anatomical landmark and a frame of reference, the actions include automatically selecting one or more series of MR images from a plurality of series of MR images previously acquired with the MR scanner and having the anatomical landmark and the frame of reference. The actions also include automatically selecting one or more candidate groups from the one or more series of MR images that were selected, wherein selection of the one or more candidate groups is based on predetermined rules. The actions further include automatically selecting a respective reference image from each candidate group of the one or more candidate groups that were selected, wherein selection of each respective reference image is based on the predetermined rules. The actions still further include automatically displaying the respective reference image selected for each candidate group of the one or more candidate groups that were selected, wherein each respective reference image is displayed in reference viewports on a user interface on a display based on the predetermined rules.

In a further embodiment, a non-transitory computer-readable medium, the computer-readable medium including processor-executable code that when executed by a processing system including one or more processors, causes the processing system to perform actions. For a given magnetic resonance (MR) image acquired with an MR scanner for an MR scan and having an anatomical landmark and a frame of reference, the actions include automatically selecting one or more series of MR images from a plurality of series of MR images previously acquired with the MR scanner and having the anatomical landmark and the frame of reference. The actions also include automatically selecting one or more candidate groups from the one or more series of MR images that were selected, wherein selection of the one or more candidate groups is based on predetermined rules. The actions further include automatically selecting a respective reference image from each candidate group of the one or more candidate groups that were selected, wherein selection of each respective reference image is based on the predetermined rules. The actions still further include automatically displaying the respective reference image selected for each candidate group of the one or more candidate groups that were selected, wherein each respective reference image is displayed in reference viewports on a user interface on a display based on the predetermined rules.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 is a block diagram of an MRI apparatus, in accordance with aspects of the present disclosure;

FIG. 2 is a schematic diagram of a scan control device of the MRI apparatus in FIG. 1, in accordance with aspects of the present disclosure;

FIG. 3 is a swimlane diagram of a timeline of events carried out by the scan control device of FIG. 2 during a contrast scan, in accordance with aspects of the present disclosure;

FIG. 4 is a schematic diagram of a scan interface in a task planning view (e.g., during a first state during a first stage of a contrast scan), in accordance with aspects of the present disclosure;

FIG. 5 is a schematic diagram of a scan interface in a live scanning view (e.g., during a second state during a second stage of a contrast scan), in accordance with aspects of the present disclosure;

FIG. 6 depicts a selected coronal reference image on the left for an axial bolus observation plane image, in accordance with aspects of the present disclosure;

FIG. 7 depicts a selected sagittal reference image on the left for an axial bolus observation plane image, in accordance with aspects of the present disclosure;

FIG. 8 illustrates a flow diagram of a method for selecting reference images for a contrast-enhanced magnetic resonance scan, in accordance with aspects of the present disclosure;

FIG. 9 illustrates a schematic diagram of a three-dimensional depiction for finding a slice from a candidate group which has a center nearest to a center of a BOP slice, in accordance with aspects of the present disclosure;

FIG. 10 illustrates a schematic diagram of a two-dimensional depiction for finding a slice from a candidate group which has a center nearest to a center of a BOP slice, in accordance with aspects of the present disclosure;

FIG. 11 is a schematic diagram of a mathematical formulation of a vector projection approach, in accordance with aspects of the present disclosure;

FIG. 12 illustrates a flow diagram of a method for selecting a reference image using an optimized Euclidean distance approach, in accordance with aspects of the present disclosure;

FIG. 13 illustrates a flow diagram of a method for selecting a reference image using a standard Euclidean distance approach, in accordance with aspects of the present disclosure;

FIG. 14 depicts a table illustrating a complexity comparison for various approaches for selecting a reference image, in accordance with aspects of the present disclosure;

FIG. 15 illustrates a flow diagram of a method for selecting a reference image (e.g., utilizing an orientation-based grouping logic), in accordance with aspects of the present disclosure; and

FIG. 16 illustrates a flow diagram of a method for determining an approach to utilize for reference image selection based on image slice positions, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

The following provides a list of terms and definitions as used in the disclosure. The term “orientation” refers to one of three anatomical orthogonal planes: axial, sagittal, or coronal. The orientation of an oblique (not a true orthogonal) image in this disclosure refers to the nearest approximate orthogonal orientation: one of axial, sagittal, or coronal. The term “group” refers to a subset of images in a series having the same characteristics, such as orientation, b-value, slice position, or other characteristics. The term “series” refers to images scanned with one pulse sequence and sharing the same Series Instance UID DICOM attribute. The term “GRx” refers to three graphic presentation viewports in the MR console, which can be used to facilitate the preparation of a prescription by providing the user an intuitive graphical interface-on which the prescribed slices and their exact relations with the examined organ can be visualized using an already scanned image as a reference image. The term “candidate group” refers to a series or its subset selected based on some pre-defined rules, from which reference images are selected.

The following description relates to medical imaging workflows, and in particular contrast-enhanced magnetic resonance (MR) workflows or other types of MR workflows. Contrast-enhanced MR imaging (MRI) scans include the administration of a paramagnetic contrast agent, such as gadolinium-based contrast agents, to an imaging subject in order to enhance contrast of various tissues of the imaging subject. Contrast-enhanced MRI scans demand that imaging start at the beginning or peak level of contrast enhancement in a region of interest (such as an artery). A typical workflow for a contrast-enhanced MRI scan includes an estimation of the time for a contrast bolus of the contrast agent to travel from the injection site to the region of interest (ROI) and the acquisition is started by an operator of the MRI system in the hopes that the operator has the timing correct. To estimate the time from when the contrast bolus is injected to when the contrast bolus arrives at the ROI, low-resolution live two-dimensional (2D) images of the ROI may be displayed, and the operator may trigger the start of the post-contrast acquisitions (which may include acquisitions in order to generate higher resolution, three-dimensional (3D) images) based on the viewed contrast intensity of the ROI via the live 2D images. In other examples, the MRI system may track/plot the contrast intensity of the ROI over time and the MRI system or the operator may trigger the start of the post-contrast acquisitions based on the plot reaching a threshold value.

However, each of these approaches has drawbacks. For example, some imaging subjects may never exhibit a contrast level in the ROI that is high enough to reach the threshold for triggering the start of the post-contrast acquisitions. Further, relying solely on the live 2D images may result in inadvertent timing errors (e.g., delays) in triggering the post-contrast acquisitions due to operator distractions or misinterpretation of the contrast level in the live 2D images. Thus, it may be desirable for the operator of the MRI system to be able to view both the contrast plot and the live 2D images to make an informed decision, and it may be further desirable for the MRI system to provide a back-up timing for automatically triggering the start of the post-contrast acquisitions to avoid missed scans due to unusual contrast kinetics of the imaging subject, for example. With current MRI systems, the live 2D images and contrast plot are not be displayed simultaneously, and monitoring both the live 2D images and the contrast plot may demand the operator switch between different interfaces or toggle between different views, which may further exacerbate the issues mentioned above by increasing the cognitive load placed on the operator and increasing the likelihood the operator may be become distracted, which may increase the likelihood that the correct timing for initiating the contrast scan acquisitions will be missed.

Thus, according to embodiments disclosed herein, a scan interface may be displayed that gives the operator of the MRI system a real-time, live view of the contrast scan process by allowing the operator to visualize the arrival of the contrast bolus via both live 2D images and an automatically-generated, live contrast plot that are displayed simultaneously in one interface, combined with multiple methods of triggering the contrast scan acquisitions, including an operator-initiated trigger and an auto/system-initiated trigger, each of which may include a back-up trigger based on a time since injection of the contrast bolus. The scan interface may further allow a scan prescription for the contrast scan to be set via an easy-to-visualize timeline, as well as allow operator adjustments to the ROI for triggering the post-contrast scan and other parameters. These functionalities may be presented on a single interface displayed on a display associated with a computing device (e.g., a scan control device), which may provide a specific manner of displaying a limited set of information (e.g., the live 2D images, contrast plot, scan prescription timeline, and/or various user interface controls selectable to adjust scan parameters) to the user, rather than using conventional user interface methods to display a generic index/list on a computer that may require the user to step through multiple menus and/or interfaces of images, plots, and scan prescription information to find the relevant contrast level information and scan prescription settings. The scan interface disclosed herein may be advantageous because it avoids a user having to scroll around and switch views/interfaces multiple times to find desired data/functionality, thereby preventing drilling down through many layers to get the desired data/functionality which may be slow, complex, and difficult to learn. The disclosed scan interface may improve the efficiency of using the computing device by bringing together the scan information most relevant to the user, allowing the user to view the most relevant scan information without accessing separate interfaces, menus, or display panels where contrast level information and scan prescription settings may be displayed. The speed of a user's navigation through various views and windows may be improved because the disclosed scan interface saves the user from navigating to separate interfaces or display panels to enable the contrast level or scan prescription settings to be seen or a function of interest to be activated.

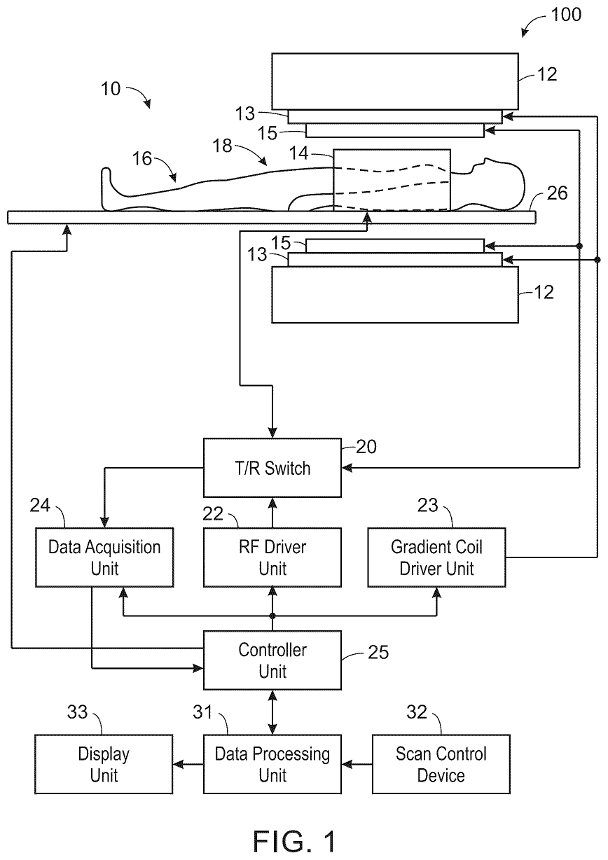

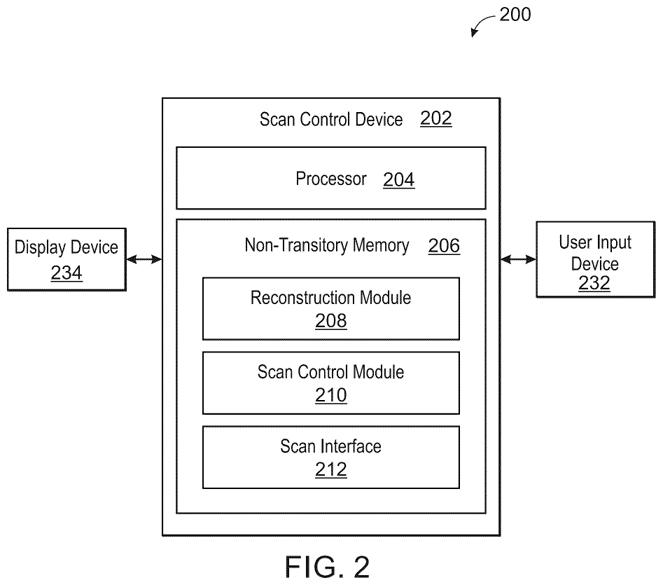

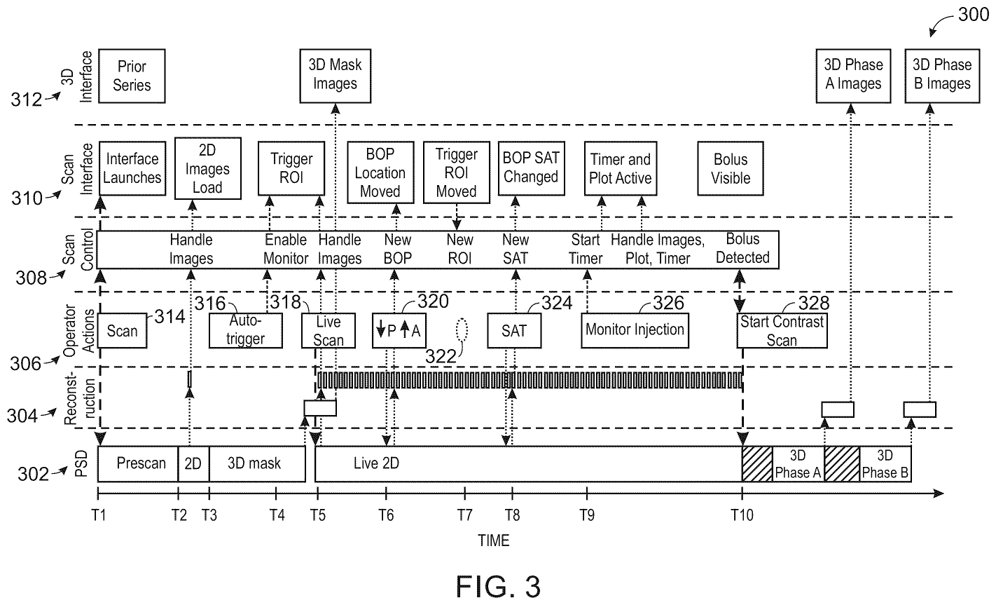

An example MRI apparatus that may be used to obtain images of an imaging subject during a contrast-enhanced MRI scan is shown in FIG. 1. The MRI apparatus may include a scan control device, such as the scan control device of FIG. 2, configured to process data from the MRI apparatus to form images, command actions of the MRI apparatus (e.g., start the contrast scan acquisitions), and display a scan interface to enable a user to monitor a contrast level of an imaging subject during the scan, set scan prescription parameters, and the like. The scan control device may carry out actions as dictated by the user, via the scan interface, to control the MRI apparatus during the contrast scan, such as according to the timeline shown in FIG. 3. During the course of the contrast scan, the scan interface may be in various states depending on the stage of the contrast scan.

FIG. 1 illustrates an MRI apparatus 10 (e.g., an MRI system) that includes a magnetostatic field magnet unit 12, a gradient coil unit 13, an RF coil unit 14, an RF body coil unit 15 (e.g., volume coil unit), a transmit/receive (T/R) switch 20, an RF driver unit 22, a gradient coil driver unit 23, a data acquisition unit 24, a controller unit 25, a patient bed or table 26, a data processing unit 31, a scan control device 32, and a display unit 33. In some embodiments, the RF coil unit 14 is a surface coil, which is a local coil typically placed proximate to the anatomy of interest of a subject 16. Herein, the RF body coil unit 15 is a transmit coil that transmits RF signals, and the local surface of the RF coil unit 14 receives the MR signals. As such, the transmit body coil (e.g., RF body coil unit 15) and the surface receive coil (e.g., RF coil unit 14) are separate but electromagnetically coupled components. The MRI apparatus 10 transmits electromagnetic pulse signals to the subject 16 placed in an imaging space 18 with a static magnetic field formed to perform a scan for obtaining magnetic resonance signals from the subject 16. One or more images of the subject 16 can be reconstructed based on the magnetic resonance signals thus obtained by the scan.

The magnetostatic field magnet unit 12 includes, for example, an annular superconducting magnet, which is mounted within a toroidal vacuum vessel. The magnet defines a cylindrical space surrounding the subject 16 and generates a constant primary magnetostatic field B₀.

The MRI apparatus 10 also includes a gradient coil unit 13 that forms a gradient magnetic field in the imaging space 18 so as to provide the magnetic resonance signals received by the RF coil arrays with three-dimensional positional information. The gradient coil unit 13 includes three gradient coil systems, each of which generates a gradient magnetic field along one of three spatial axes perpendicular to each other, and generates a gradient field in each of a frequency encoding direction, a phase encoding direction, and a slice selection direction in accordance with the imaging condition. More specifically, the gradient coil unit 13 applies a gradient field in the slice selection direction (or scan direction) of the subject 16, to select the slice; and the RF body coil unit 15 or the local RF coil arrays may transmit an RF pulse to a selected slice of the subject 16. The gradient coil unit 13 also applies a gradient field in the phase encoding direction of the subject 16 to phase encode the magnetic resonance signals from the slice excited by the RF pulse. The gradient coil unit 13 then applies a gradient field in the frequency encoding direction of the subject 16 to frequency encode the magnetic resonance signals from the slice excited by the RF pulse.

The RF coil unit 14 is disposed, for example, to enclose the region to be imaged of the subject 16. In some examples, the RF coil unit 14 may be referred to as the surface coil or the receive coil. In the static magnetic field space or imaging space 18 where a static magnetic field Bo is formed by the magnetostatic field magnet unit 12, the RF body coil unit 15 transmits, based on a control signal from the controller unit 25, an RF pulse that is an electromagnet wave to the subject 16 and thereby generates a high-frequency magnetic field B₁. This excites a spin of protons in the slice to be imaged of the subject 16. The RF coil unit 14 receives, as a magnetic resonance signal, the electromagnetic wave generated when the proton spin thus excited in the slice to be imaged of the subject 16 returns into alignment with the initial magnetization vector. In some embodiments, the RF coil unit 14 may transmit the RF pulse and receive the MR signal. In other embodiments, the RF coil unit 14 may only be used for receiving the MR signals, but not transmitting the RF pulse.

The RF body coil unit 15 is disposed, for example, to enclose the imaging space 18, and produces RF magnetic field pulses orthogonal to the main magnetic field B₀ produced by the magnetostatic field magnet unit 12 within the imaging space 18 to excite the nuclei. In contrast to the RF coil unit 14, which may be disconnected from the MRI apparatus 10 and replaced with another RF coil unit, the RF body coil unit 15 is fixedly attached and connected to the MRI apparatus 10. Furthermore, whereas local coils such as the RF coil unit 14 can transmit to or receive signals from only a localized region of the subject 16, the RF body coil unit 15 generally has a larger coverage area. The RF body coil unit 15 may be used to transmit or receive signals to the whole body of the subject 16, for example. Using receive-only local coils and transmit body coils provides a uniform RF excitation and good image uniformity at the expense of high RF power deposited in the subject. For a transmit-receive local coil, the local coil provides the RF excitation to the region of interest and receives the MR signal, thereby decreasing the RF power deposited in the subject. It should be appreciated that the particular use of the RF coil unit 14 and/or the RF body coil unit 15 depends on the imaging application.

The T/R switch 20 can selectively electrically connect the RF body coil unit 15 to the data acquisition unit 24 when operating in receive mode, and to the RF driver unit 22 when operating in transmit mode. Similarly, the T/R switch 20 can selectively electrically connect the RF coil unit 14 to the data acquisition unit 24 when the RF coil unit 14 operates in receive mode, and to the RF driver unit 22 when operating in transmit mode. When the RF coil unit 14 and the RF body coil unit 15 are both used in a single scan, for example if the RF coil unit 14 is configured to receive MR signals and the RF body coil unit 15 is configured to transmit RF signals, then the T/R switch 20 may direct control signals from the RF driver unit 22 to the RF body coil unit 15 while directing received MR signals from the RF coil unit 14 to the data acquisition unit 24. The coils of the RF body coil unit 15 may be configured to operate in a transmit-only mode or a transmit-receive mode. The coils of the RF coil unit 14 may be configured to operate in a transmit-receive mode or a receive-only mode.

The RF driver unit 22 includes a gate modulator (not shown), an RF power amplifier (not shown), and an RF oscillator (not shown) that are used to drive the RF coils (e.g., RF body coil unit 15) and form a high-frequency magnetic field in the imaging space 18. The RF driver unit 22 modulates, based on a control signal from the controller unit 25 and using the gate modulator, the RF signal received from the RF oscillator into a signal of predetermined timing having a predetermined envelope. The RF signal modulated by the gate modulator is amplified by the RF power amplifier and then output to the RF body coil unit 15.

The gradient coil driver unit 23 drives the gradient coil unit 13 based on a control signal from the controller unit 25 and thereby generates a gradient magnetic field in the imaging space 18. The gradient coil driver unit 23 includes three systems of driver circuits (not shown) corresponding to the three gradient coil systems included in the gradient coil unit 13.

The data acquisition unit 24 includes a pre-amplifier (not shown), a phase detector (not shown), and an analog/digital converter (not shown) used to acquire the magnetic resonance signals received by the RF coil unit 14. In the data acquisition unit 24, the phase detector phase detects, using the output from the RF oscillator of the RF driver unit 22 as a reference signal, the magnetic resonance signals received from the RF coil unit 14 and amplified by the pre-amplifier, and outputs the phase-detected analog magnetic resonance signals to the analog/digital converter for conversion into digital signals. The digital signals thus obtained are output to the data processing unit 31.

The MRI apparatus 10 includes a table 26 for placing the subject 16 thereon. The subject 16 may be moved inside and outside the imaging space 18 by moving the table 26 based on control signals from the controller unit 25.

The controller unit 25 includes a computer and a recording medium on which a program to be executed by the computer is recorded. The program when executed by the computer causes various parts of the apparatus to carry out operations corresponding to predetermined scanning. The recording medium may comprise, for example, a ROM, flexible disk, hard disk, optical disk, magneto-optical disk, CD-ROM, or non-volatile memory card. The controller unit 25 is connected to the scan control device 32 and processes the operation signals input to the scan control device 32 and furthermore controls the table 26, RF driver unit 22, gradient coil driver unit 23, and data acquisition unit 24 by outputting control signals to them. The controller unit 25 also controls, to obtain a desired image, the data processing unit 31 and the display unit 33 based on operation signals received from the scan control device 32.

The scan control device 32 includes user input devices such as a touchscreen, keyboard and a mouse. The scan control device 32 is used by an operator, for example, to input such data as an imaging protocol and to set a region where an imaging sequence is to be executed. The data about the imaging protocol and the imaging sequence execution region are output to the controller unit 25.

The data processing unit 31 includes a computer and a recording medium on which a program to be executed by the computer to perform predetermined data processing is recorded. The data processing unit 31 is connected to the controller unit 25 and performs data processing based on control signals received from the controller unit 25. The data processing unit 31 is also connected to the data acquisition unit 24 and generates spectrum data by applying various image processing operations to the magnetic resonance signals output from the data acquisition unit 24.

The display unit 33 includes a display device and displays an image on the display screen of the display device based on control signals received from the controller unit 25. The display unit 33 displays, for example, an image regarding an input item about which the operator inputs operation data from the scan control device 32. The display unit 33 also displays a two-dimensional (2D) slice image or three-dimensional (3D) image of the subject 16 generated by the data processing unit 31.

During an MRI scan using the MRI apparatus 10, a subject may be positioned within the imaging space 18 and an acquisition protocol may be carried out to obtain MR signals of the subject. The acquisition protocol may include a plurality of pulse sequences where in each pulse sequence, contrast is prepared via one or more RF pulses applied by the RF body coil unit 15 and the gradient coil unit 13 is controlled to spatially encode the resultant MR signals. The spatially-encoded MR signals are received by the RF coil unit 14 are digitized and stored in k-space. Thus, k-space data or a k-space dataset may refer to the raw MR signals prior to processing into an image. In some examples, one line of k-space may be filled with the raw MR signals per pulse sequence (also referred to as repetition time). In other examples, one line of k-space may be filled with the raw MR signals per echo, where more than one echo is generated per pulse sequence/repetition time. The k-space data may also be referred to as imaging data or MR data herein.

Referring to FIG. 2, scan control device 202 configured to control scan parameters of an MRI scan is shown. In some embodiments, scan control device 202 is incorporated into the MRI apparatus 10. For example, scan control device 202 may be provided in the MRI apparatus 10 as scan control device 32. In some embodiments, at least a portion of scan control device 202 is disposed at a device (e.g., edge device, server, etc.) communicably coupled to the MRI apparatus 10 via wired and/or wireless connections. In some embodiments, at least a portion of scan control device 202 is disposed at a separate device (e.g., a workstation) which can communicate with the controller unit of the MRI apparatus, for example. Scan control device 202 may be operably/communicatively coupled to a user input device 232 and a display device 234. In some examples, the user input device 232 may be the user input device of scan control device 32, explained above. Likewise, display device 234 may be the display unit 33 of MRI apparatus 10.

Scan control device 202 includes one or more processors, such as processor 204, configured to execute machine readable instructions stored in non-transitory memory 206. Processor 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Non-transitory memory 206 may store a reconstruction module 208, a scan control module 210, and a scan interface 212. Reconstruction module 208 may be configured to reconstruct images from k-space data. In some examples, reconstruction module 208 may be the data processing unit 31 of FIG. 1, such that the data processing unit and scan control device are integrated into a single device. However, in other examples, reconstruction module 208 may be omitted and scan control device 202 may be in communication with the data processing unit 31 to obtain images for display.

Scan control module 210 may be configured to send commands to the MRI apparatus (e.g., to controller unit 25) in order to control aspects of a scan carried out by the MRI apparatus. Scan control module 210 may control aspects of the scan based on user input, which may be received via the scan interface 212, in some examples. For example, the scan interface 212 may include a scan prescription display panel via which a user may set parameters for the scan (e.g., the number of phases, delay time between phases). The scan interface 212 may further include various scan control buttons, such as a live scan button that, when selected by a user, is configured to trigger acquisition and display (on the scan interface 212) of live 2D images and a start contrast scan button that, when selected, is configured to trigger acquisition of post-contrast (e.g., 3D) images. Scan control module 210 may process the acquired live 2D images in order to measure contrast level in an ROI, and the scan interface 212 may display a plot of the measured contrast level over time. Additional details about the scan interface 212 and scan control module 210, including what is displayed via the scan interface 212 during a contrast scan and what actions are taken by the scan control module 210 during the contrast scan, are provided below with respect to FIG. 3.

In some embodiments, non-transitory memory 206 may include components disposed at two or more devices, which may be remotely located and/or configured for coordinated processing. In some embodiments, one or more aspects of non-transitory memory 206 may include remotely-accessible networked storage devices configured in a cloud computing configuration.

User input device 232 may comprise one or more of a touchscreen, a keyboard, a mouse, a trackpad, a motion sensing camera, or other device configured to enable a user to interact with and manipulate data within scan control device 202. In one example, user input device 232 may enable a user to make a selection of a scan protocol, adjust scan prescription settings, select or adjust a contrast-tracking region, and the like, as well as initiate, pause, and adjust scanning.

Display device 234 may include one or more display devices utilizing virtually any type of technology. In some embodiments, display device 234 may comprise a computer monitor, and may display MR images, including images reconstructed by reconstruction module 208. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view MRI images produced by an MRI system, and/or interact with various data stored in non-transitory memory 206.

It should be understood that scan control device 202 shown in FIG. 2 is for illustration, not for limitation. Another appropriate image processing system may include more, fewer, or different components.

FIG. 3 is an example swimlane diagram 300 showing actions/events that may occur during a contrast scan of a subject using the MRI apparatus of FIG. 1, including the scan control device of FIG. 2. Diagram 300 depicts events/actions over time, with time depicted on the horizontal axis, for a plurality of aspects of the MRI apparatus that are depicted along the vertical axis, including the actions taken by the MRI apparatus (and particular the MRI scanner) in lane 302 (specifically, a pulse sequence diagram (labeled “PSD” in FIG. 3) that depicts the pulse sequence carried out by the scanner to facilitate image acquisition), the image reconstruction module in lane 304 (labeled “Reconstruction” in FIG. 3), operator interactions with the scan interface in lane 306 (labeled “Operator actions”), actions taken by the scan control module (“Scan control”) in lane 308, the display status/state of the scan interface in lane 310 (“Scan interface”), and the display status/state of a 3D interface (“3D interface”) configured to display acquired 3D images in lane 312. The image reconstruction module may perform both 2D image reconstruction and 3D image reconstruction, and 2D image reconstruction is depicted in lane 304 of FIG. 3 as the small rectangles while 3D image reconstruction is depicted in lane 304 as the larger rectangles positioned immediately below the 2D image reconstruction. Communication between devices/modules (e.g., between the scan control module and the controller unit of the MRI apparatus) is shown via the vertical arrows in FIG. 3. Time points of interest are depicted along the horizontal axis. It is to be appreciated that the term “MRI scanner” or “scanner” as used herein may refer to the components of the MRI apparatus that are activated to produce and receive MR signals (such as the magnetostatic field magnet unit 12, the gradient coil unit 13, the RF coil unit 14, and the RF body coil unit 15), as well as the associated control components (e.g., the RF driver unit 22, the gradient coil driver unit 23, the data acquisition unit 24, and the controller unit 25).

The contrast scan may commence at time TI in response to operator selection of a Scan button 314 on the scan interface or a separate interface. Selection of the Scan button 314 causes the scan control module to command the scanner to initiate a prescan pulse sequence, which may include various subject-specific calibrations of the MRI apparatus. Additionally, selection of the Scan button 314 causes the scan interface to be launched. The scan interface, when initially launched, may include viewports for displaying 2D images, a scan prescription display panel, and various user interface elements to facilitate selection of a contrast observation slice/plane for observing contrast enhancement, a trigger ROI for measuring the contrast level, and so forth. The slice/plane for observing contrast enhancement may be referred to as the bolus observation plane (BOP). Because imaging has yet to commence, no images are initially displayed in the scan interface. Via the scan prescription display panel, the operator may set parameters for the scan, such as the number of 3D contrast phases, whether or not 3D mask images are to be obtained, delays or pauses in the scan, and so forth. Optionally, prior images of the subject may be displayed in the 3D interface.

After the prescan phase, the scanner may perform an initial 2D acquisition (e.g., of one or more series) at time T2, which may result in reconstruction of a single 2D image in each of three scan planes (e.g., axial, sagittal, and coronal). The scan control module may receive the initial 2D images and load the 2D images into the viewports of the scan interface. The operator may indicate the anatomy of interest to be scanned via placement of a first box/rectangle over the displayed 2D images. The operator may also indicate the BOP (e.g., the anatomical region for observing the contrast level of the subject) via a second box/rectangle over the displayed 2D images. Once the operator selects/indicates the BOP, the image in that plane may be displayed in a main viewport (referred as the BOP viewport) and the other images (which show the orthogonal views) may be displayed in smaller reference viewports. After the initial 2D acquisition, the scanner may perform 3D mask imaging at time T3, in the background (e.g., without changing what is displayed via the scan interface), and the 3D mask images may be displayed in the 3D interface as the 3D mask images are reconstructed. The 3D mask images are images of the anatomy acquired prior to contrast administration for use in comparison and for creating subtracted images which layer the pre-and post-contrast images to show differences.

While the 3D mask imaging is occurring, the operator may interact with the scan interface to enable or disable auto-triggering and set the trigger ROI. Auto-triggering may include the automatic initiation of the post-contrast 3D acquisitions (e.g., the MR signal acquisition following administration of a contrast bolus for generation of diagnostic images), based on an automatic measurement of the contrast level in the subject following administration of the contrast bolus. The trigger ROI may be the area of the BOP that is evaluated in order to automatically measure the contrast level in the subject. For example, as shown in FIG. 3, the operator may select a user interface button (e.g., an auto-triggering button 316) at time T4 to enable auto-triggering monitoring, and a trigger ROI may be placed on the images displayed on the scan interface. The trigger ROI may be initially placed in a default location, and the operator may move the trigger ROI, resize the trigger ROI, etc. Further, it is to be appreciated that the operator may enable or disable auto-triggering at any point prior to the start of the post-contrast 3D acquisitions.

After the 3D mask imaging is complete, the scanner may pause until the operator initiates live 2D scanning. The operator may initiate live 2D scanning by selecting a live scanning button, e.g., a Start Live Scan button 318 of the scan interface, at time T5. Once the Start Live Scan button 318 is selected, the scan control module may command the scanner to commence live 2D scanning. During live 2D scanning, the scanner may perform acquisitions of MR signals (referred to as live 2D acquisitions) that are reconstructed into low resolution, 2D images. The live 2D acquisitions may be performed at a relatively high rate and the 2D images reconstructed from the MR signals may be displayed in real-time, as the images are reconstructed, in the viewports of the scan interface. In some examples, the 2D images may be displayed at a frame rate of two frames per second, although other frame rates are possible without departing from the scope of this disclosure.

As described in greater detail below, in certain embodiments, the scan control module automatically selects the references images to be displayed in the viewports and utilized in determining the BOP (e.g., after the initial 2D acquisition and/or during the live 2D scanning). The workflow for automatic selection of reference images eliminates the burden on the user to choose reference images for BOP live scan manually, thus, reducing the overall time required for the workflow and improving productivity.

While the live 2D scanning is occurring, the operator may interact with the scan interface to make various adjustments, if desired. For example, at time T6, the operator may move the BOP plane in an anterior or posterior direction, for example, via BOP movement elements 320 (e.g., a line and/or arrows that are placed over the reference images in the reference viewports). If the operator adjusts the BOP plane, the scan control module notifies the scanner to change the slice that is being acquired, and the slice of the images displayed in the BOP viewport changes accordingly. In some examples, the operator may move the trigger ROI at time T7, by dragging the trigger ROI 322 that is displayed on the live 2D images and/or by selecting a trigger marker button and then entering keystrokes (e.g., side arrows, up/down arrows) to move the trigger ROI. In still further examples, the operator may enable or disable saturation (SAT) at time T8 for the BOP image by selecting a SAT button 324 on the scan interface. Other adjustments are possible as well, such as enabling or disabling image subtraction for the BOP image. Because the measurement of the contrast level includes a measurement of the baseline, non-contrast pixel brightness in the BOP image at the trigger ROI (e.g., for comparison as the brightness increases with contrast uptake), adjusting the BOP location, saturation, trigger ROI location/size, or image subtraction entails acquisition of a new baseline, non-contrast BOP image (e.g., new 3D mask images).

Once the operator has set the scan prescription and is satisfied with the BOP location, auto-triggering settings, and is otherwise ready for the 3D contrast acquisitions to begin, the contrast bolus is injected to the subject. Upon initiation of the injection of the contrast agent, the operator selects a monitoring button, such as the Monitor Injection button 326, of the scan interface, such as at time T9. The selection of the Monitor Injection button 326 triggers the scan control module to start a back-up timer. Additionally, the selection of the Monitor Injection button 326 also triggers the scan control module to start measuring the contrast level in the trigger ROI and generate a plot of the measured contrast level over time. Both the timer and the plot are displayed in the scan interface and are updated at a relatively high rate, e.g., at the frame rate of the live 2D imaging. As such, the contrast plot may be a live plot that includes a real-time curve of contrast intensity over time. Live 2D scanning is ongoing and the live 2D images are displayed in the scan interface. As the contrast bolus arrives at the trigger ROI, the brightness of the BOP image, at least at the trigger ROI, increases and the contrast bolus becomes visible on the scan interface, which is visually indicated via both the live 2D images and the contrast plot.

The post-contrast scan commences in response to any of three possible triggers. If auto-triggering is enabled, the post-contrast scan commences once the measured contrast level reaches a threshold level. Alternatively, for example if auto-triggering is not enabled, the post-contrast scan may commence once the operator selects a contrast scanning button, such as the Start Contrast Scan button 328, of the scan interface. In either example, the post-contrast scan may commence once the back-up timer reaches a threshold duration, if the threshold duration is reached before the measured contrast level reaches the threshold or before the operator selects the Start Contrast Scan button 328. The post-contrast scan may include one or more phases of 3D image acquisition optionally separated by delays. For example, FIG. 3 shows the post-contrast scan commencing at time T10, where the post-contrast scan includes two image-acquisition phases (3D phase A and 3D phase B), with the first phase commencing after a delay and the second phase commencing after the first phase and an intervening delay. At least in some examples, the pulse sequences carried out for the post-contrast scan may be different than the pulse sequences carried out to acquire the live 2D images. Once the 3D images have been reconstructed, the 3D post-contrast images are displayed via the 3D interface.

The techniques described herein may be utilized with the techniques for contrast-enhanced scanning described in U.S. patent application Ser. No. 18/499,043, entitled “SYSTEMS AND METHOD FOR CONTRAST IMAGING”, filed Oct. 31, 2023, and U.S. patent application Ser. No. 18/499,055, entitled “SYSTEMS AND METHOD FOR CONTRAST IMAGING”, filed Oct. 31, 2023, which are herein incorporated by reference in their entirety. Although discussed in the context of MRI, the techniques described herein may also be utilized with other imaging techniques (e.g., computed tomography imaging).

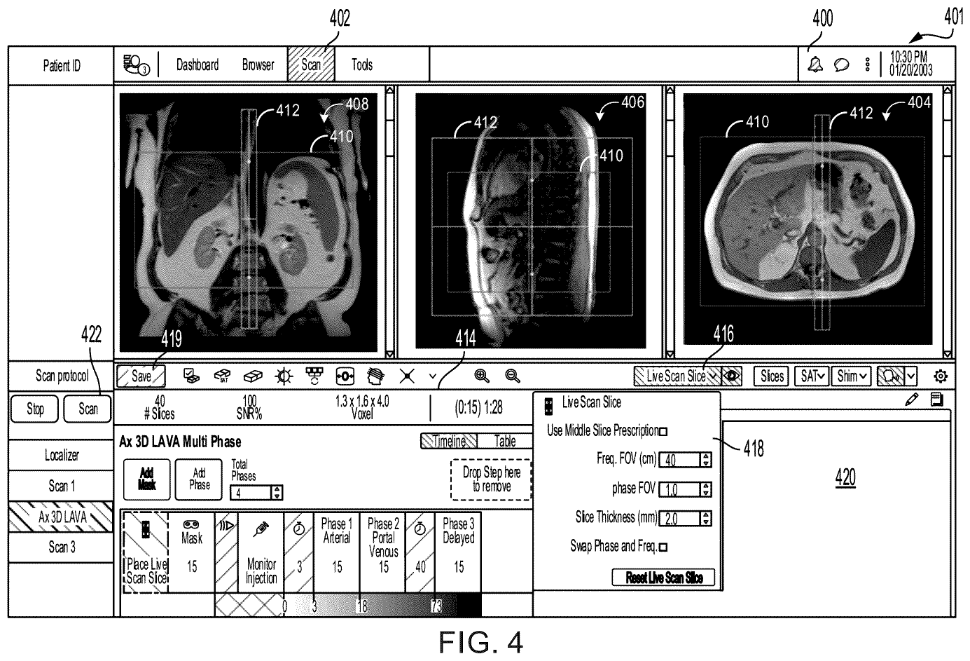

FIG. 4 shows an example of a scan interface 400 in a task planning (e.g., prescription) view 402. The scan interface 400 (e.g., user interface) may be the scan interface 212 of FIG. 2 and, thus, may include features discussed above with respect to FIG. 3. The scan interface 400 may be displayed after the initial 2D image acquisition (e.g. of one or more series) has occurred and before live 2D scanning has commenced (e.g., between times T2 and T5 of FIG. 3). The scan interface 400 in the task planning view 401 may be displayed in response to user selection of a scan tab 402. A first image 404 is displayed in a first viewport (e.g., GRx viewport), a second image 406 is displayed in a second viewport (e.g., GRx viewport), and a third image 408 is displayed in a third viewport (e.g., GRx viewport). In the example shown, the first image 404 is an axial view, the second image 406 is a sagittal view, and the third image 408 is a coronal view, each reconstructed from the k-space data acquired during the initial 2D acquisition. A user (e.g., the operator of the MRI apparatus (e.g., a technician)) may prescribe the field of view (e.g., anatomy of interest) to be scanned in the contrast scan by placing/moving/resizing a first box 410 onto each of the three images. The contrast scan being prescribed in the example shown herein is a liver acceleration volume acquisition (LAVA) scan, and thus the first box 410 is placed to scan the liver. Further, the operator may place/move/resize a second box 412 onto an anatomical feature (e.g., a vessel) of each of the three images to designate a slice for contrast tracking (also referred to as the BOP or contrast observation slice). Based on the placement of the second box 412, the BOP slice/contrast observation slice is determined. The scan interface 400 in the task planning view 401 further includes a live scan slice menu 416, which when selected causes a live scan slice display panel 418 to be displayed. Via the live scan slice display panel 418, the operator may make adjustments to the live scan slice (e.g., the BOP slice), such as the frequency and phase FOVs and slice thickness.

A user (e.g., the operator of the MRI apparatus (e.g., a technician)) may prescribe the field of view (e.g., anatomy of interest) to be scanned in the contrast scan by placing/moving/resizing a first box 422 onto each of the three images 414, 418, and 420. The box 422 is also displayed relative to the BOP 411 in the GRx viewports 406, 408, and 410. The contrast scan being prescribed in the example shown herein is a liver acceleration volume acquisition (LAVA) scan, and thus the first box 422 is placed to scan the liver. Further, the operator may place/move/resize a second box 424 onto an anatomical feature (e.g., a vessel) of each of the three images 414, 418, and 420 to designate a slice for contrast tracking (also referred to as the BOP 411 for contrast observation slice). Based on the placement of the second box 424, the BOP slice/contrast observation slice is determined.

The scan interface 400 in the prescription view 401 further includes a scan prescription display panel 414. The scan prescription display panel 414 may include various user interface elements that may be selected or adjusted in order to set or edit the number of 3D scanning phases and associated delays of the scan prescription for carrying out the contrast scan. In the example shown in FIG. 4, the timeline view of the scan prescription display panel is shown, with the phases and delays depicted as a function of time. Once the operator is finished editing the scan prescription, the operator may select a Save button 419, which may save the changes made to the scan prescription. The scan interface 400 in the task planning view 401 may also include an instructions panel 420 wherein instructions, tips, etc., for interacting with the scan interface 400 may be displayed. For example, in the task planning view 402, the instructions panel 420 may instruct the operator on the purpose of the first box 410, the second box 412, the live scan slice menu 416, and the scan prescription display panel 414. As described in greater detail below, in certain embodiments, the scan control module automatically selects the references images to be displayed in the viewports and utilized in determining the BOP.

The scan interface 400 in the task planning view 401 includes additional panels, such as patient information panels (e.g., including patient identifying information such as name and/or patient ID) and a scan protocol panel. The scan protocol panel may list the scans that are to be carried out on the patient, such as a localizer scan, the contrast scan, and/or any other scans (e.g., a screening scan, such as a single-shot fast spin echo), and the scans listed may change in visual appearance to indicate which scans have already been carried out, which is the current scan, and which scans are left to carry out. The scan protocol panel may further include a scan button 422. Selection of the scan button 422 may launch a live scanning view of the scan interface, described in more detail below. It is to be appreciated that the scan button 422 may be positioned elsewhere on the scan interface without departing from the scope of this disclosure.

Thus, the task planning view 401 of the scan interface 400 allows for the operator to prescribe or place, prior to being shown the live view of the patient's anatomy, a rectangle to indicate to the system which anatomical region is of interest. Via the prescription view of the scan interface, the operator may also construct a timeline-based series of phases, or steps, that they want the exam to use, referred to as the scan prescription.

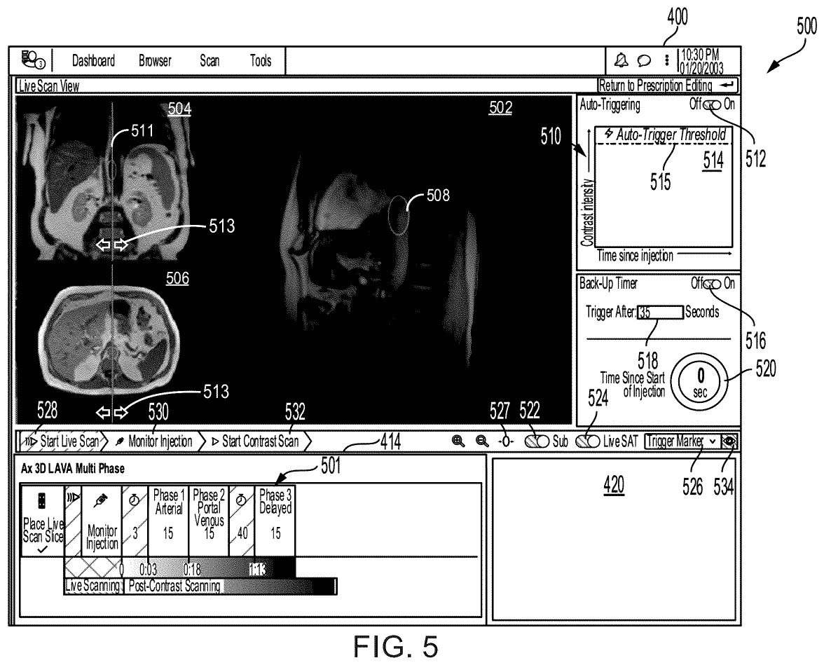

FIG. 5 shows a live scanning view 500 of the scan interface 400. The scan interface 400 may be displayed in the live scanning view 500 after the second box 412 has been placed and thus the BOP has been designated, after the operator has saved (e.g., by selecting the Save button 419 in FIG. 4) any changes made to the scan prescription via the scan prescription display panel 414, and after the operator has selected the scan button 422 in FIG. 4. The live scanning view 500 may initially show the previously acquired 2D images, until the operator initiates live scanning via a Start Live Scan button 528. For example, upon selection of the Save button 419 in the task planning view 401 in FIG. 4, the scan interface 400 may be updated to show the live scanning view 500 of the scan prescription and some of the interface elements may be replaced with new interface elements, including the Start Live Scan button 528. Once selected, the Start Live Scan button 502 may change in visual appearance (e.g., change color) to indicate that the scan is currently in the live 2D scanning phase. The scanning view 501 may include a timeline that visually indicates any earlier steps of the scan prescription that are complete (e.g., via a check mark) as well as visually indicate the current step of the scan prescription.

Thus, the live scanning view 500 may be a live, real-time scanning view wherein the images displayed are updated in real-time as new images are reconstructed from continuously-acquired k-space data. The images are displayed in a main, BOP viewport 502, a first reference viewport 504, and a second reference viewport 506. As depicted, the BOP viewport 502 is larger than first reference viewport 504 and the second reference viewport 506 to emphasize the BOP/contrast observation slice. As depicted, the first reference viewport 504 and the second reference viewport 506 is located to the left of the BOP viewport 502. The first reference viewport 504 is located above the second reference viewport 506. The BOP viewport 502 may include images of the BOP (e.g., the contrast observation slice), as explained previously, and the reference viewports 504, 506 may include images in the planes orthogonal to the BOP. For example, the BOP viewport 502 shows a live image in the sagittal plane, the first reference viewport 504 shows an image in the coronal plane, and the second reference viewport 56 shows an image in the axial plane.

In the live scanning view 500, the scan interface 400 further includes a contrast tracking display panel 510 that includes an auto-triggering button 512. In the example shown in FIG. 5, the operator has toggled the auto-triggering button 512 to the on position, thereby enabling auto-triggering. As a result, a trigger ROI 508 has been placed on the image in the BOP viewport 502, as well as on the images in the reference viewports 504, 506. The operator may move the trigger ROI 508 by dragging the trigger ROI 508, which causes the corresponding trigger ROI on the images in the reference viewports to move as well. The reference viewports 504 and 506 include a respective line 511 (e.g., cross-reference line) indicating the position/orientation of the BOP. The two arrows 513 at the end of the cross-reference lines 511 can be clicked by the user to move the BOP to a different plane parallel to the current one (e.g., if the operator clicks on the arrow 513 pointing right, the BOP/contrast observation slice may be adjusted to be one slice to the right of the current slice). It should be noted it is always preferable to show reference images orthogonal to the BOP so that the cross-reference lines 511 can effectively visualized and consequently movement of the arrows 513 can be effectively used.

The contrast tracking display panel 510 includes a contrast plot 514 that is configured to display, after live scanning has commenced and after injection of the contrast bolus, measured contrast level (e.g., intensity) as a function of time since contrast injection. The contrast plot 514 includes an auto-trigger threshold 515 that may be adjusted by the operator (e.g., via dragging the auto-trigger threshold 515 up or down). As further described herein, when the measured contrast level equals or exceeds the auto-trigger threshold 515 and auto-triggering is enabled, a post-contrast scan including one or more post-contrast acquisitions may be initiated.

The contrast tracking display panel 510 further includes interface elements for enabling and setting a back-up timer. The back-up timer may be enabled via a back-up timer button 516 and the duration of the back-up timer may be adjusted/set by the operator via a timer duration menu 518. The timer duration menu 518 may include a box for text entry, as shown, or the timer duration menu 518 may include toggle buttons or a drop-down menu whereby the operator may select a duration from among a plurality of preset durations. When the back-up timer is enabled, the back-up timer may begin counting responsive to receiving an indication (e.g., via selection of a Monitor Injection button 530) that a contrast bolus has been injected to the imaging subject. Thus, the contrast tracking display panel 510 further includes a timer 520 that may visually indicate the time since the start of the injection.

The scan interface 400 in the live scanning view 500 includes a plurality of interface elements that may be selected/actuated in order to adjust scan settings and/or trigger various actions. For example, the live scanning view 500 includes a subtraction button 522 that the operator may toggle on or off to enable or disable image subtraction, respectively. Likewise, the live scanning view 500 includes a SAT button 524 that the operator may toggle on or off to enable or disable saturation, respectively. The live scanning view 500 additionally includes a Trigger Marker menu 526. When selected, the Trigger Marker menu 526 causes an additional menu/display panel to be displayed, via which the operator may make adjustments to the trigger ROI 508. For example, the Trigger Marker menu 526 may include inputs for adjusting the length and width of the trigger ROI numerically. The operator may also adjust those values of the trigger ROI (e.g., length and width) directly in the BOP viewport 502 by dragging anchor points on the trigger ROI 508. The anchor points may only be visible when the trigger ROI 508 is selected. Additionally, the scan interface may include a trigger icon 534 that, when selected, causes the trigger ROI 508 to be displayed and, when deselected, causes the trigger ROI 508 to be removed from the BOP viewport. Further, in some examples, the live scanning view 500 may include a reset to default button 527 that may be selected in order to revert certain settings, such as zoom level, window width/length, etc.

Further, the live scanning view 500 includes the Start Live Scan button 528, the Monitor Injection button 530, and a Start Contrast Scan button 532. Once the injection of the contrast bolus has begun, the operator may select the Monitor Injection button 530 to indicate to the scan control module to begin measuring the contrast level in the trigger ROI 508 (which would be displayed in the contrast plot 514) and start the back-up timer (which would be shown via the timer 520). If the auto-triggering is not enabled, the operator may select the Start Contrast Scan button 532 to signal to the scan control module to commence the post-contrast 3D scan (e.g., start the 3D acquisitions following any specified delay). In some examples, even if the operator does not enable auto-triggering, the contrast level may still be tracked and displayed via the contrast tracking display panel 510, which may allow the operator to make an informed decision about when to initiate the post-contrast scan. In other examples, if the operator does not enable auto-triggering, the contrast level may not be tracked, which may increase the efficiency of the scan control device by reducing the processing demands associated with tracking the contrast level. In an example, a method may include a first scan with auto-triggering, and then a second scan without auto-triggering, via the same imaging system and same user interface and protocols as described herein.

The scan interface 400 in the live scanning view 500 may also include the instructions panel 420 wherein instructions, tips, etc., for interacting with the scan interface 400 may be displayed. For example, in the live scanning view 500, the instructions panel 420 may instruct the operator on the purpose of the trigger marker, the subtraction and SAT buttons, and the Monitor Injection button.

Thus, the scan interface 400 in the live scanning view 500 provides a real-time view that may be displayed after the operator has prescribed the scan and has pressed the “start scan” button. This view includes: a constantly updating live view of the patient's anatomy; orthogonal reference viewports (two smaller viewports); a graph (upper right) showing how much contrast the scanner is registering while “watching” the region of interest; a timer (middle right) that acts as a back-up for both the operator and the system missing the identification of the bolus arrival; and a timeline that shows the operator exactly where the scanner is in the scan process. As mentioned above, the large viewport in the center is a real-time, updating view of the patient's anatomy. The two smaller viewports are orthogonal views of the same region. The line and arrows in the small reference viewports allow the operator to shift the view of the large, live view by discrete amounts.

The reference viewports 504 and 506 include a respective line 518 (e.g., cross-reference line) indicating the position/orientation of the BOP 512. The two arrows 520 at the end of the cross-reference lines 518 can be clicked by the user to move the BOP 512 to a different plane parallel to the current one. It should be noted it is always preferable to show reference images orthogonal to the BOP 512 so that the cross-reference lines 518 can effectively visualized and consequently movement of the arrows 520 can be effectively used.

Upon adjustment of the BOP 512, the patient can be administered the contrast agent and then the user can click or select a Monitor Injection button 522. Once the presence of contrast is determined to be peaking in the region of interest, the user can trigger the 3D contrast scan manually by clicking or selecting a Start Contrast Scan button 524. Alternatively, the 3D contrast scan may be automatically triggered by using a backup trigger time via Back-Up Timer feature 526 is turned on.

During task planning, although the user may have planned on one image, the user may have chosen to scroll past the image in GRx, before starting the task, so there is no reliable way to determine the exact image(s) on which the user planned. Moreover, the GRx viewports may contain images from multiple series, which may or may not have the required orthogonal orientations among them. The workflows described herein in which the images to be displayed as reference images are automatically selected by the application (e.g., scan control module) for any given GRx planning use-case, provide a consistent, reliable, and efficient visualization to the user, which in turn enables the user to select an accurate image location (e.g., BOP) to inject and track the contrast in real time. The disclosed embodiments can also be utilized to solve a more generalized problem: wherein for a given MR image (e.g., BOP image), the most suitable reference images are automatically selected from one or more previously scanned series with the same landmark (e.g., anatomical landmark) based on a set of predefined rules (e.g., orthogonality, minimum distance, etc.).

The disclosed workflow eliminates the burden on the user to manually chose reference images for BOP live scan, thus, reducing the overall time required for the workflow and improving productivity. The disclosed embodiments (which have a near-zero learning curve) enable a user to perform the contrast-enhanced scan with minimum clicks, which would be beneficial for relatively less experienced technologists. The user is allowed to scroll through the reference viewports to find a more suitable reference image (e.g., the image on which the user planned, if desired). This provides more flexibility to change the reference image from what is computed or selected from the disclosed workflow, thus, avoiding the need for a repeat scan.

The scan control module via the viewports handles the image visualization functionalities. One of the main processing logics during initialization involves selection of reference images from the series loaded in the GRx viewports or form one or more series previously scanned with same frame of reference, and displaying them in the reference viewports. The following is example set of predefined rules for selecting reference images (e.g., using images from GRx viewports).

First, among the predefined rules, the images (e.g., reference images) should be orthogonal to the orientation of the BOP image (when possible). This is the primary criteria for selection of candidate groups. The other secondary criteria can be slice position, b-value, angle, SNR, etc., in case of there being multiple orthogonal groups available. This is because the reference viewports together with the cross-reference lines (e.g., the lines 518 and the arrows 520 in FIG. 5) enable the user to accurately visualize the BOP and also move the BOP to a new parallel plane if required.

Second, among the predefined rules, images (e.g., reference images) shall be selected from a non-localizer series whenever possible. Previously, when a localizer series is scanned, it automatically populated the GRx viewports, and if the user wanted to use any other series in the GRx viewports for planning, they to manually select and add them. This meant that the images available in the GRx viewports until then were not sufficient to do accurate planning and the newly added images (e.g., from a non-localizer series) are preferred for accurate planning. Hence, these are given higher priority over localizer images and are automatically loaded in GRx viewports.

Third, among the predefined rules, images (e.g., reference images) with their center closest to the BOP image center shall be selected for display. This ensures the best visualization of cross-reference lines in the reference viewports across all possible BOP locations throughout the scan.

Fourth, among the predefined rules, if one of the predefined orientations is not available in the series loaded in the GRx viewports, then images with the other orientation are loaded into both of the reference viewports.

Fifth, among the predefined rules, if none of the series loaded in the GRx viewports has an orientation orthogonal to the Bop orientation, then images with the same orientation as that of the BOP shall be loaded in the reference viewports, and the cross-reference lines are not displayed.

Sixth, among the predefined rules, the top reference viewport may always display an axial, sagittal, or coronal image in that priority order from given selected reference images, based on their availability compared to the bottom viewport. For example, if the two selected reference images are one sagittal image and one coronal image, then the sagittal one will always be displayed in the two viewport and the coronal image in the bottom one, and so on. The priority order may be changed via user configuration. For example, as shown in FIG. 5, the images are shown in a different order in the reference viewports.

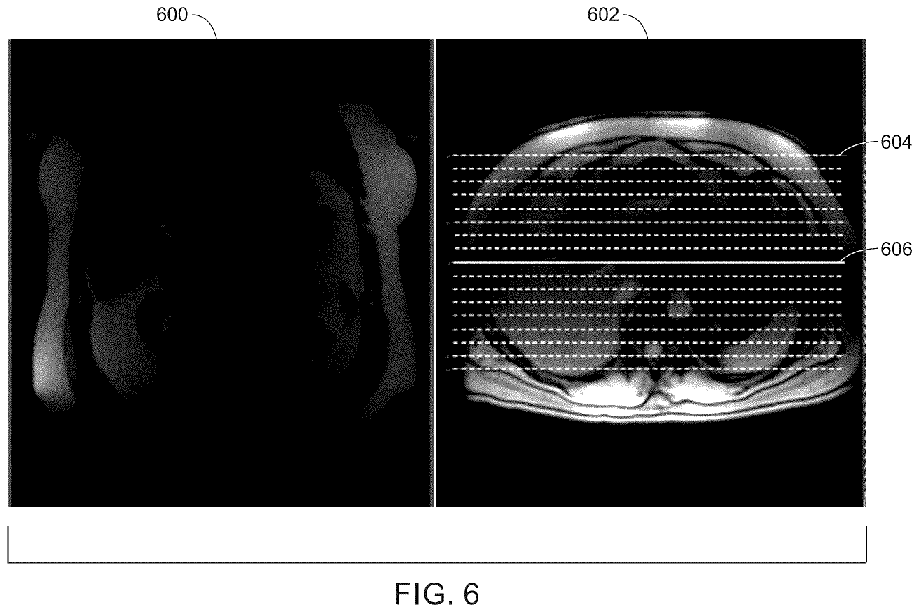

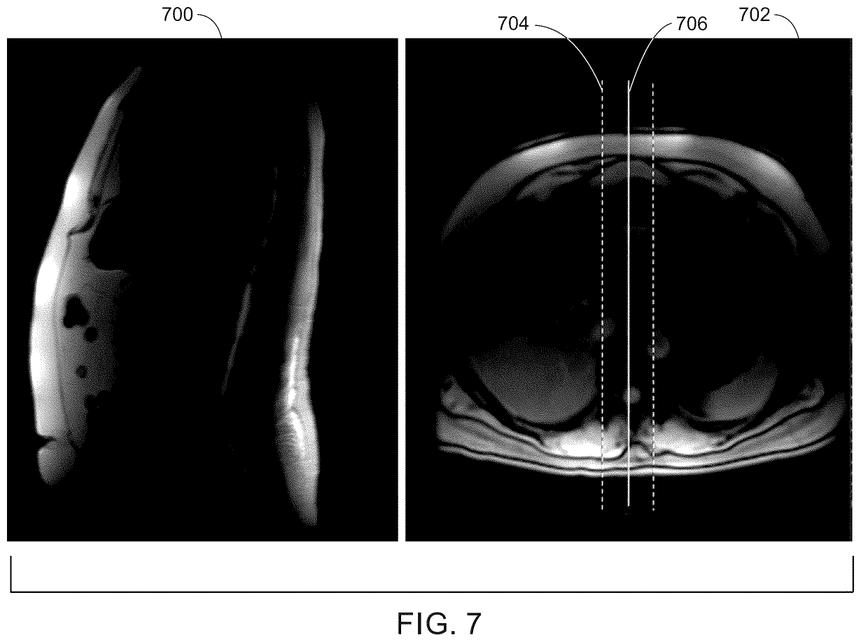

FIGS. 6 and 7 give examples of reference image selection using the set of predefined rules described above. FIG. 6 depicts a selected coronal reference image 600 for an axial BOP image 602. The selected coronal reference image 600 (e.g., selected coronal localizer image) is depicted on the left of FIG. 6. The axial BOP image 602 is depicted on the right side of FIG. 6. Dashed lines 604 on axial BOP image 602 indicated locations of coronal localizer images relative to the to the axial BOP image 602. Solid line 606 on the axial BOP image indicates the location of the selected coronal image 600 relative to the axial BOP image 602.

FIG. 7 depicts a selected sagittal reference image 700 for an axial BOP image 702. The selected sagittal reference image 700 (e.g., selected sagittal localizer image) is depicted on the left of FIG. 7. The axial BOP image 702 is depicted on the right side of FIG. 7. Dashed lines 704 on axial BOP image 702 indicated locations of sagittal localizer images relative to the to the axial BOP image 702. Solid line 706 on the axial BOP image indicates the location of the selected sagittal image 700 relative to the axial BOP image 702.

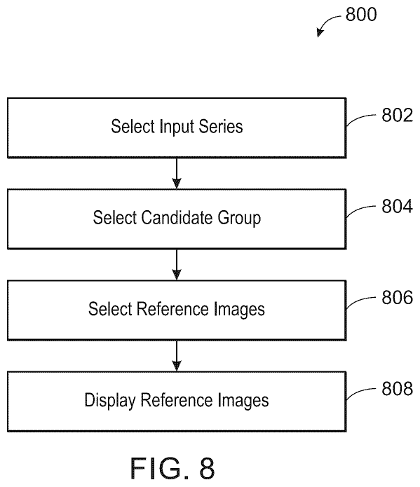

FIG. 8 illustrates a flow diagram of a method 800 for selecting reference images for a contrast-enhanced magnetic resonance scan. One or more steps of the method 800 may be performed by processing circuitry of the magnetic resonance imaging system 10 in FIG. 1. One or more of the steps of the method 800 may be performed simultaneously or in a different order from the order depicted in FIG. 8.

The steps for the method 800 are for a given MR image acquired with an MR scanner and having an anatomical landmark and a frame of reference. In certain embodiments, the given MR image is a bolus observation plane image utilized to visualize and to track an incoming contrast bolus. In certain embodiments, each respective reference image that is selected and displayed is utilized to plan or adjust a bolus observation plane. In certain embodiments, each respective reference image displays a respective cross-reference line indicating a location of the bolus observation plane image relative to the respective reference image.

The method 800 includes selecting one or more series (e.g., input series) of MR images from a plurality of series of MR images previously acquired with the MR scanner and having the anatomical landmark and the frame of reference (block 802). For example, in certain embodiments, for the given MR image (e.g., BOP image), the input series is automatically selected as the series available in GRx viewports when a task is initialized. In certain embodiments, the user interface is configured so that user can manually select one or more previously scanned series with the same landmark/frame of reference as that of the BOP image, without loading them in the GRx viewports.

The method 800 also includes automatically selecting one or more candidate groups from the one or more series (e.g., input series) of MR images that were selected, wherein selection of the one or more candidate groups is based on predetermined rules (e.g., the predetermined rules described above) (block 804). In certain embodiments, a maximum of two candidate groups are selected, since the user interface has two reference viewports. A candidate group can contain the whole of or part of one series, depending on the grouping logic utilized. Some grouping logics considered are orientation (e.g., axial, sagittal, coronal), b-value, angle, signal-to-noise ratio and other factors as described in greater detail below.

The method 800 further includes automatically selecting a respective reference image from each candidate group of the one or more candidate groups that were selected, wherein selection of each respective reference image is based on the predetermined rules (e.g., the predetermined rules described above) (block 806). In certain embodiments, a particular algorithm (as described in greater detail below) is utilized in the selecting the respective reference images. Examples of algorithms include a vector projection method, an optimized Euclidean distance method, a general Euclidean distance method, or another type of algorithm. In certain embodiments, instead of automatically selecting a respective reference image, a selection of a respective reference image from each candidate group may be received from the user via the user interface. In certain embodiments, instead of automatically selecting a respective reference image, the first image form each candidate group is selected as the respective reference image from the candidate group. In certain embodiments, after automatic selection of a respective reference image, a selection of a respective reference image from each candidate group may be received from the user via the user interface to be displayed instead of the automatically selected respective reference image being displayed.

The method 800 even further includes automatically displaying the respective reference image selected for each candidate group of the one or more candidate groups that were selected, wherein each respective reference image is displayed in reference viewports on a user interface on a display based on the predetermined rules (e.g., the predetermined rules described above) (block 808). A number of different workflows may be utilized to display the reference images which are described in greater detail below.

The following describes the selection of candidate groups from one or more input series (e.g., block 804 of the method 800 in FIG. 8). The selection of candidate groups is based on a set of predetermined rules (e.g., as given in the following). In the following it is assumed that the series information is obtained from GRx viewports are about SeriesA, SeriesB, and SeriesC. It also assumed there are three possible orientations: axial (A), sagittal(S), and coronal (C) and X∈{A,S,C} be the BOP orientation. Also, the top reference viewport image is designated I1 and the bottom viewport image is designated I2. The objective is to select I1 and I2 from SeriesA, SeriesB and SeriesC.

For the following, if the number of series equals 1 (Series A=Series B=Series C) (Step 1). If all images in the series are in a single orientation, then the series is marked as a candidate group, and the reference image Ir is determined from this group using the predefined rules described above. Then both reference viewports will have the same image, I1=I2=Ir (Step 1(a)). If the series contains 2 orientations X1,X2∈{A,S,C}, then check if {X1,X2}=={A,S,C}−{X} (Step 1(b)). If the answer is yes, then both with orientations X1 and X2 are marked as candidate groups and respective reference images Ir1 and Ir2 found. Then, I1=Ir1 and I2=Ir2 (Step 1bi). If the answer is no, then mark group with orientation equals {A,S,C}−({X}∩{X1,X2}) (i.e., the group with images orthogonal to BOP) as the candidate group and determine reference image Ir. Then I1=I2=Ir (Step 1(b) (ii)). If the series contains 3 orientations (such as a 3-plane localizer), then groups with orientations {A,S,C}−{X} (i.e., orthogonal to BOP) are marked as candidate groups and find respective reference images Ir1 and Ir2. Then, I1=Ir1, I2=Ir2 (Step 1(c)).

For the following, if the number of series equals 2, then there are SeriesA and SeriesB (Step 2). If SeriesA is of a single orientation (e.g., Xa∈{A,S,C}), and SeriesB is of multiple orientation (Step 2(a)), then if Xa does not belong to {A,S,C}−{X}, (i.e., SeriesA has images of same orientation as that of BOP), then candidate groups are determined and subsequently reference images determined from SeriesB based on the above steps 1b) or 1c) (Step 2(a)(i)). If Xa∈{A,S,C}−{X}, then mark SeriesA as a candidate group, and find reference image Ir (Step 2(a)(ii)). Find the other candidate group from SeriesB whose orientation={A,S,C}−{X,Xa}. If the candidate group exists (Step 2(a)(ii)(1)), reference image Ir2 is found. Then, I1−Ir1 and I2=Ir2. If the candidate group does not exist (Step2(a)(ii)(2), then display the image from SeriesA in both the top and bottom viewports (i.e., I1=I2=Ir1). If SeriesA and SeriesB are of multiple orientations (Step 2(b)), then find candidate groups and reference images form SeriesA using both Step1(b)(i) or Step1(c) (Step2(b)(i)). If Series A is of the case in Step1(b)(ii), then find one reference image Ir1 as in Step1(b)(ii) (Step2(b)(ii). The candidate group with the other orientation is checked in SeriesB. If found (Step2(b)(ii)(1)), then the reference image, Ir2, is found from the candidate group in SeriesB. If not found Step2(b)(ii)(2), then the reference image from Series A is displayed both reference viewports (i.e., I1=I2=Ir1). If SeriesA and SeriesB are of a single orientation, respectively, Xa, Xb∈{A,S,C} (Step2(c), then if Xa,Xb}={A,S,C}−{X}, then mark both series as candidate groups and find reference images from SeriesA and SeriesB (i.e., Ir1 and Ir2). Then, I1=Ir1 and I2=Ir2 (Step2(c)(i)). If not (Step2(c)(ii), then mark as a candidate group the series which has orientation e {A,S,C}−{X}, and find the reference image form this series. Then both reference viewports will show the same reference image, I1=Ir and I2=Ir2.

When the number of series is 3 (or more if the input set of series is not coming from GRx viewports), a combination of techniques described above is used to optimally determine the candidate group(s). The current viewport layout requires selection of 2 reference images; hence, the above techniques find 2 output images I1 and I2. The techniques can be appropriately modified to generate any number of output images, meeting specific rules.

Moreover, the above set of rules basing orientation as a grouping logic, is not exhaustive. There can be multiple other grouping logics that can be used for selection, depending on the pulse sequences. For example, these grouping logics may include b-value (e.g., for a diffusion weighted imaging sequence), phase (e.g., for a multi-phase sequence), signal-to-noise ratio (e.g., for an Inhance sequence), slice position (e.g., for a cine sequence), and angle (e.g., for an multi-slice multi-acquisition sequence). These examples are not exhaustive.

The following describes the selection of a respective reference image from a candidate group (e.g., block 806 of the method 800 in FIG. 8). For each candidate group that is available for reference image selection, the reference image is selected based on some predefined rules. One such example used is the minimum distance rule as explained below.

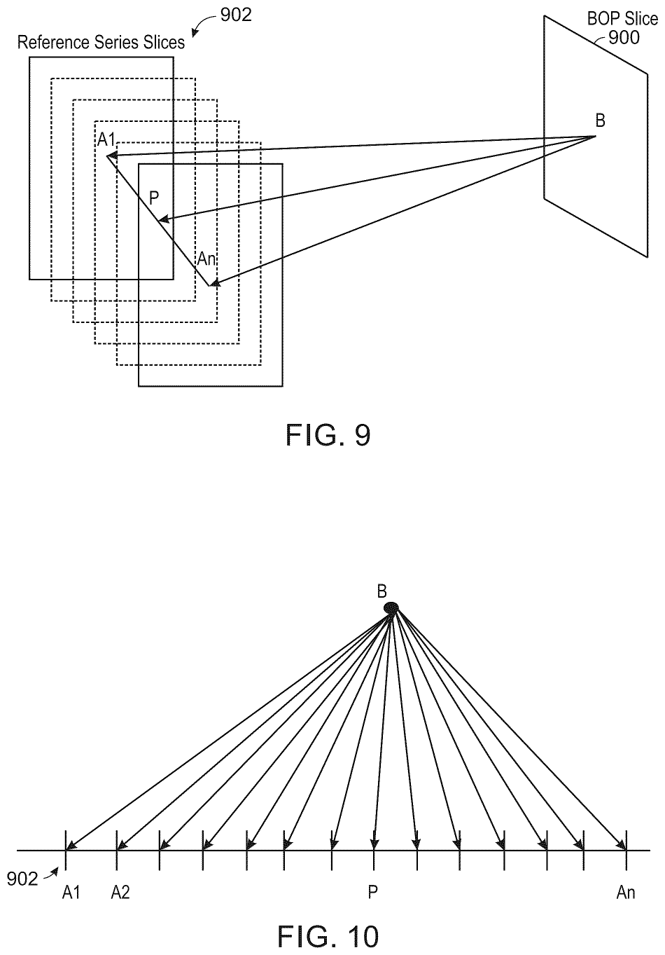

For a given candidate group having images in parallel planes, there are different slices available at multiple slice locations, and there is a BOP slice. The goal is to find the slice from the candidate group which has the center nearest to the center of the BOP slice. FIG. 9 depicts a 3D depiction of a schematic diagram for finding a slice from a candidate group which has a center nearest to a center of a BOP slice, wherein there is a BOP slice 900 present, with center B from which the closest image in the candidate group is to be determined. As depicted, all the points connecting the centers of images in a candidate group 902 (e.g., reference series slices) lie on the same line A1An, and there is a plane passing through that line and point B. FIG. 10 is the depiction of the same in 2D which consists of the line segment A1An and BOP center B. Now there are n points lying on a line segment, and the problem is to find the point P nearest to point B.

As described below, there are multiple algorithms that can be utilized to select the reference image from a candidate group. For example, a vector projection approach can be utilized to select the reference image from a given candidate group when the following conditions are met. First, the slice numbers of consecutive images are in continuous order either in an increasing or a decreasing order. Second, there are no repeating slice location in the group (i.e., no 2 images have the exact same slice position). Third, the center of all the images present in the group lie on the same line connecting the centers of first and last images in that group.

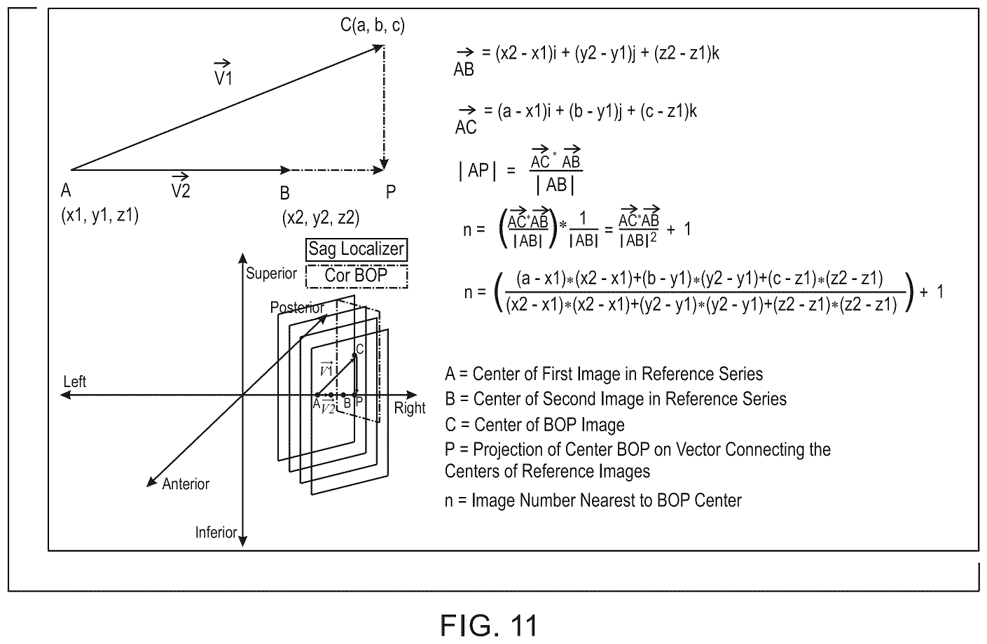

FIG. 11 is a schematic diagram of a mathematical formulation of a vector projection approach. In particular, FIG. 11 depicts the calculation of instance number of the reference image with a minimum distance from the BOP slice, once a candidate group is identified. The projection of vector V1 connecting the center of a first image in a candidate group and the center of a BOP image is taken in the direction of the vector V2 connecting the first image and a second image in the candidate group by getting the dot product and dividing it by the magnitude of vector V2 (i.e., the distance between the two images, d). Then, the projection is divided by the distance between the two images. This gives the perpendicular projection of BOP center on the line connecting all the centers of the reference images. The image closest to that projection is the image which would have the shortest distance from the center of BOP. If there is only 1 image present for one orientation in a non-localizer or localizer series, the same is considered as the reference image to be shown for that orientation in the respective reference viewport.

Other points to note, include 1 is added to the final result because the number calculated indicates the number of images between the selected reference image and the first image. Also, a negative dot product indicates that the BOP lies to the left of the first image. In that case, the very first image will be the image with least distance from the BOP center, and hence the selected reference image. Further, if the calculated image number n is greater than the last image number present in the series, then the last image will be the image with least distance from the BOP center, and hence the selected reference image. Even further, if the slice numbers are in decreasing order, the selected reference image number will be equal to the computed image number calculated subtracted from the total number of images in the series.

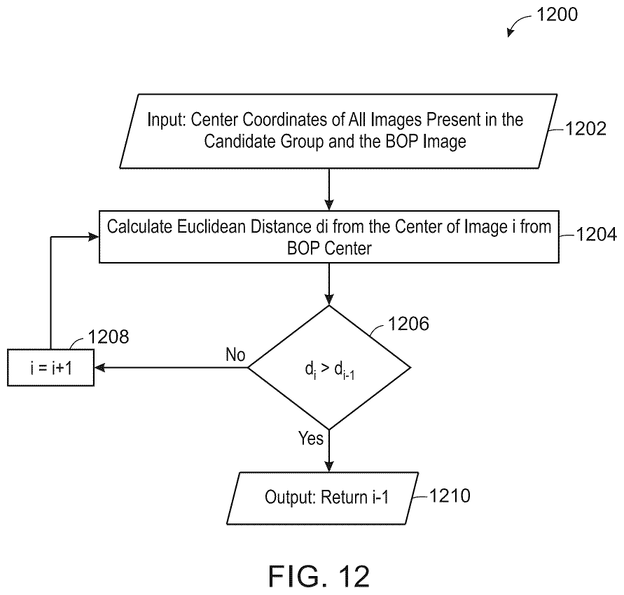

In certain embodiments, the optimized Euclidean approach can be utilized to compute the reference image form a given candidate group when the centers of all the images present in the candidate group lie on the same line connecting the centers of the first and last images in that group. FIG. 12 illustrates a flow diagram of a method 1200 for selecting a reference image using an optimized Euclidean distance approach. One or more steps of the method 1200 may be performed by processing circuitry of the magnetic resonance imaging system 10 in FIG. 1. One or more of the steps of the method 1200 may be performed simultaneously or in a different order from the order depicted in FIG. 12.

The method 1200 includes inputting the center coordinates of all images present in the candidate group and for the BOP image (block 1202). The method 1200 includes calculating the Euclidean distance between the center of an image in the candidate group and center of the BOP image (e.g., starting with the first image) (block 1204). The method 1200 also includes comparing each respective Euclidean distance value with the previous one (i.e., Euclidean distance for the previous image) to determine if the respective Euclidean distance value is greater than the previous one (block 1206). If the respective Euclidean distance value is not greater than the previous one, then the method 1200 continues onto the next image (block 1208) and repeats blocks 1204 and 1206. If the respective Euclidean distance value is greater than the previous one, then the method 1200 includes outputting (i.e., selecting) the previous image as the reference image of the candidate group as it has the minimum distance (block 1210) and the further computations are stopped.

In certain embodiments, a default selection can be utilized to compute the reference image when neither of the above approaches are deemed fit to compute the reference image from a candidate group or when the choice has to be made solely based on the user preference. This approach completely eliminates any computational overhead and selects the first image from the candidate group by default as the reference image. This approach is particularly suitable when the given candidate group has only one image or if it has all images in the same slice position such as in a cine sequence when the above two approaches are unsuitable, since all images have the same distance from the BOP image. The configuration of this approach can also be modified to select any image other than the first image to be the reference image, based on a predefined rule or dynamically based on user preference.

For the above approaches that are based on center coordinates of the center pixel of each image. In cases, where the candidate group has all the images with the same center coordinates but different top left coordinates (e.g., as in a radial acquisition), the top left coordinates shall be used instead of center coordinates for all computations. While some of these approaches may have certain conditions under which each approach is preferred, a user may choose to change this preference.

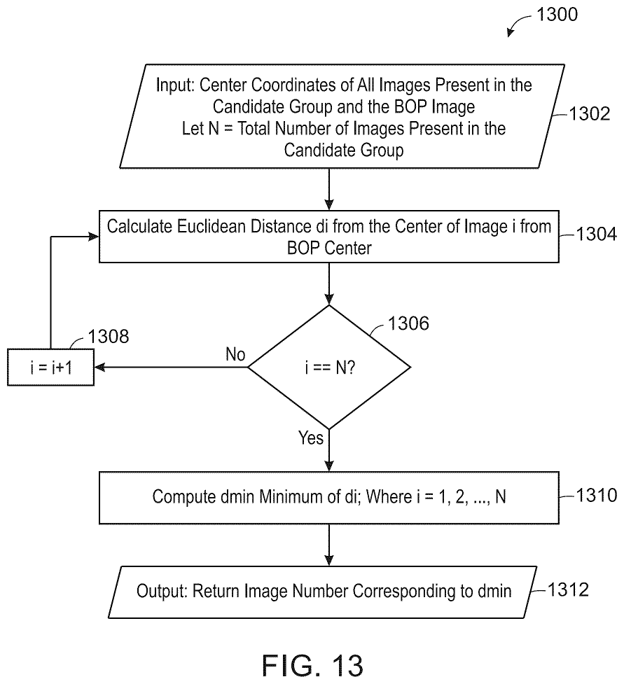

In certain embodiments, a standard Euclidean approach can be utilized to select the reference image form a given candidate group when the centers of all the images do not lie on the same line. FIG. 13 illustrates a flow diagram of a method 1300 for selecting a reference image using a standard Euclidean distance approach. One or more steps of the method 1300 may be performed by processing circuitry of the magnetic resonance imaging system 10 in FIG. 1. One or more of the steps of the method 1300 may be performed simultaneously or in a different order from the order depicted in FIG. 13.

The method 1300 inputting the center coordinates of all images present in the candidate group and for the BOP image (block 1302). N represents the total number of images present in the candidate group. The method 1300 includes calculating the Euclidean distance between the center of an image in the candidate group and center of the BOP image (e.g., starting with the first image) (block 1304). The method 1300 also includes determining if the image that the Euclidean distance was just calculated for is the last of the total number of images (block 1302). If the image that the Euclidean distance was just calculated for is not the last image, the method 1300 continues onto the next image (block 1308) and repeats blocks 1304 and 1306. If the image that the Euclidean distance was just calculated for is the last image, then the method 1300 includes determining the image that has the least distance, dₘᵢₙ, from the BOP (block 1310). The distance between the image center of each image is calculated form the BOP center while keeping track of the image number having minimum distance. The method 1300 includes outputting (i.e., selecting) the image hast the least distance, dₘᵢₙ, as the reference image of the candidate group (block 1312). The method 1300 may be utilized when the other approaches or algorithms above are not compatible with the given reference group.

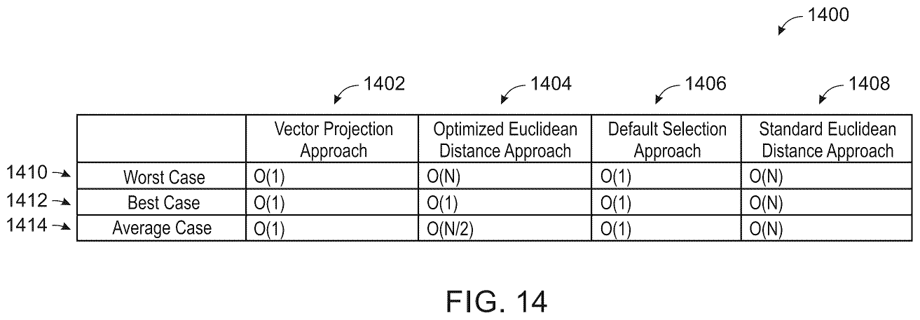

FIG. 14 depicts a table 1400 illustrating a complexity (e.g., time complexity) comparison for various approaches for selecting a reference image. The different approaches that are compared are the vector projection approach in column 1402, the optimized Euclidean distance approach in column 1404, the default selection approach in column 1406, and the standard Euclidean approach in column 1408. A top row 1410 is a worst case, a middle row 1412 is a best case, and a bottom row 1414 is an average case. The table 140 compares the time complexities for all the approaches so that a user can choose the least complex approach, if desired, based on the prerequisite conditions met by candidate groups.

Once the reference images are selected, a number of workflows may be utilized for displaying them. These workflows are for each viewport. In a first workflow, the image selected based on the predetermined workflows (e.g., blocks 804 and 806 of the method 800 in FIG. 8) is displayed. In as second workflow, the image from the GRx belonging to the candidate group's series that was available at the start of the task is displayed. In the second workflow, it is not required that block 806 of the method 800 in FIG. 8 be utilized to select the reference image. In a third workflow, the image based on user preference is displayed. For the example, the user may choose this image from any input series scanned with the same landmark as the given image (e.g., BOP image). The user may also make their choice before (e.g., through a selection mechanism provided in the user interface) or after the viewports are displayed (e.g., by dragging and dropping the image to reference viewport). In the third workflow, it is not that blocks 804 or 806 of the method 800 in FIG. 8 be utilized.

In a fourth workflow, the image from the first workflow is displayed but flexibility is given to the user for scrolling through the entire series. In a fifth workflow, the image from the second workflow is displayed but flexibility is given to the user for scrolling through the entire series. In a sixth workflow, the image from the third workflow is displayed but flexibility is given to the user for scrolling through the entire series.

In a seventh workflow, two images, one from the first workflow and the other from the second workflow, are displayed but flexibility is given to the user for scrolling to one or the other. In an eighth workflow, two images, one from the second workflow and the other from the third workflow, are displayed but flexibility is given to the user for scrolling to one or the other. In a ninth workflow, two images, one from the first workflow and the other from the third workflow, are displayed but flexibility is given to the user for scrolling to one or the other. In a tenth workflow, three images, one from the first workflow, one from the second workflow, and one from the third workflow, are displayed but flexibility is given to the user for scrolling from one to the other.

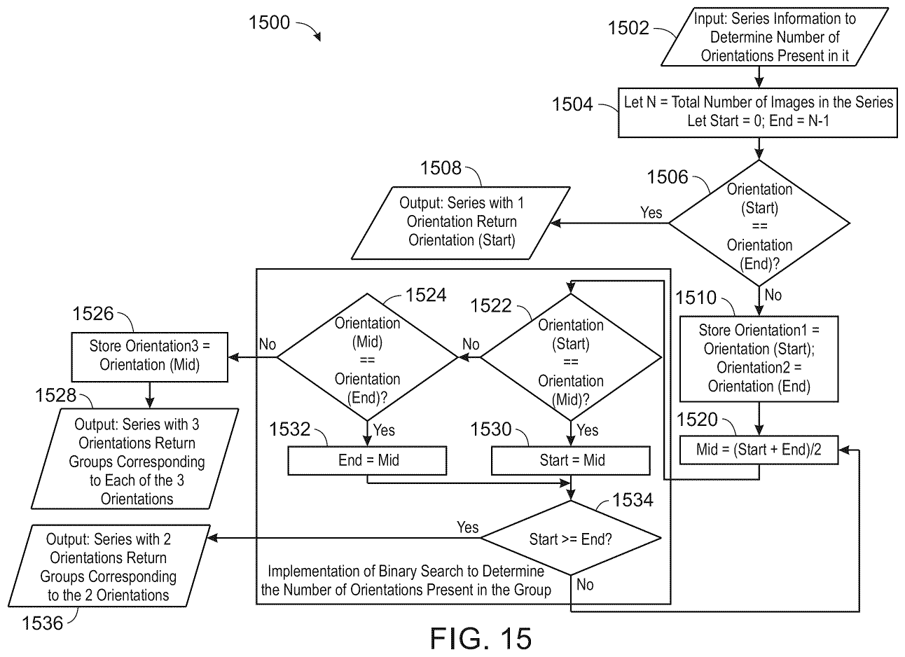

FIG. 15 illustrates a flow diagram of a use-case of the proposed method 1500 for selecting a reference image (e.g., utilizing an orientation-based grouping logic). One or more steps of the method 1500 may be performed by processing circuitry of the magnetic resonance imaging system 10 in FIG. 1. One or more of the steps of the method 1500 may be performed simultaneously or in a different order from the order depicted in FIG. 15. In the method 1550, the input series is the series available in the GRx viewports at the time of initialization of the detecting and tracking of the arrival of the contrast bolus. Also, the expected output is to find two reference images conforming the set of prescribed rules described above. The grouping logic is based on orientation. Since the 3-plane localizer is a recurring sequence in GRx viewports, used in planning the task, one or two candidates are selected based on the workflow describe in block 804 of the method 800 in FIG. 3. This step is further optimized using the method 1500.

The method 1500 includes inputting the series information to determine the number of orientations in it (block 1502). In the method 1500, let N equal the total number of images in the series with the start equaling 0 and the end equaling N−1 (block 1504). The method 1500 includes determining the orientation of the first and last images of the series have the same orientation (block 1506). If the first and last images of the series are the same orientation, then the method 1500 includes outputting that the series has one orientation (block 1508). If the first and last images of the series are not the same orientation, the method 1500 includes storing the first image as one orientation and the last image as another orientation (block 1510). If the first and last images of the series are not the same orientation, the method 1500 includes determining the middle image of the series (block 1520). If it is determined that the series is a 3-plane localizer, a binary search is utilized (e.g., as depicted in FIG. 15) to determine the number of orientations present in the group (as well as the start of the new orientation), which makes the grouping more optimal. The method 1500 includes comparing the orientation of the middle image to the orientation of the first image (block 1522). If the middle image does not have the same orientation as the first image, then the method 1500 includes comparing the orientation of the middle image to the orientation of the last image (block 1524). It the middle image does not have the same orientation as the last image, the method 1500 includes storing the orientation of the middle image as the third image (block 1526). The method 1500 then includes outputting a series with 3 orientations and returning groups corresponding to each of the 3 orientations (block 1528). If either the first image and the middle image have the same orientation (block 1530) or the last image and the middle image have the same orientation (block 1532), then the method 1500 includes determining if the orientation of the first image and the last image are the same (block 1534). If not, then the method 1500 returns to block 1520. If the orientation of the first image and the last image are the same, then the method 1500 includes outputting a series with 2 orientations and returning groups corresponding to the 2 orientations.

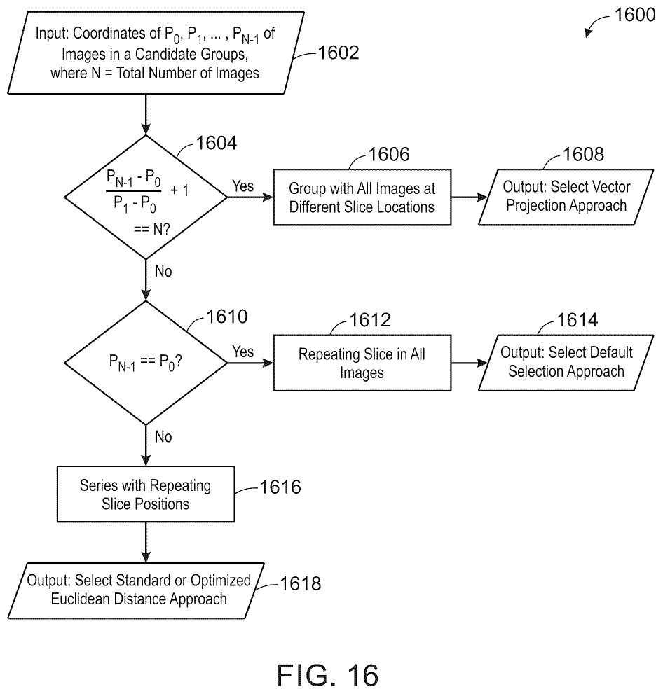

Once the candidate groups are determined, reference images are selected based on the predefined rules described above. In particular, the reference image is selected using one of algorithms described above. FIG. 16 illustrates a flow diagram of a method 1600 for determining an approach to utilize for reference image selection based on image slice positions. One or more steps of the method 1600 may be performed by processing circuitry of the magnetic resonance imaging system 10 in FIG. 1. One or more of the steps of the method 1600 may be performed simultaneously or in a different order from the order depicted in FIG. 16.

The method 1600 includes inputting the center coordinates of all of the images in a candidate group (block 1602). The method 1600 also includes determining if the distance between the center pixel coordinates of first image (P₀) and the last image (P_{N-1}) is a multiple of that of the of the first image (P_{O}) and the second image (P₁) (block 1604). If it is a multiple, the method 1600 includes grouping with all the images at different slice locations since there is a high probability that all images in the candidate group have a unique slice position and lie on the same line (block 1606). In this case, the method 1600 includes outputting a selection of the vector projection approach for selecting the reference image (block 1608).

If it is not a multiple, the method 1600 includes determining if the center pixel coordinates of the first image (P_{O}) and the last image (P_{N-1}) are the same (block 1610). If the center pixel coordinates of the first image (P_{O}) and the last image (P_{N-1}) are the same, the method 1600 includes determining that there is high probability that all images in the sequence have the same slice position (i.e., repeating slice in all images) (block 1612). In this case, the method 1600 includes outputting a selection of the default selection approach for selecting the reference image (block 1614).

If the center pixel coordinates of the first image (P_{O}) and the last image (P_{N-1}) are not the same, the method 1600 includes determining that the images are a series with repeating slice positions (block 1616). In this case, the method 1600 includes outputting a selection of either standard or optimized Euclidean distance approach (block 1618). Once the reference images are selected, then they are displayed in reference viewports of the user interface.

Technical effects of the disclosed subject matter include providing a workflow that enables selection (e.g., automatic selection) of reference images for all possible known use cases of MR sequences that can be used for planning a contrast-enhanced dynamic MR sequence using GRx viewports. Technical effects of the disclosed embodiments include providing an algorithm that uses multiple approaches based on the series involved to ensure the lowest complexity for any given type, as opposed to standard techniques, thus, improving speed and throughput. Technical effects of the disclosed subject matter include providing an approach that automates selection of reference images, without any need for manual intervention, which provides results that are consistent, reliable, less error prone, and significantly reduces or eliminates repeat scans. Technical effects of the disclosed embodiments include providing an algorithm that does not require any additional computation resources than what is already in the MR console. Technical effects of the disclosed embodiments include providing workflows that are modality agnostic and that can be applied to other modalities such as computed tomography imaging with suitable modifications.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for selecting reference images for a magnetic resonance (MR) scan, comprising:
for a given MR image acquired with an MR scanner and having an anatomical landmark and a frame of reference:
automatically selecting, via a processing system comprising one or more processors, one or more series of MR images from a plurality of series of MR images previously acquired with the MR scanner and having the anatomical landmark and the frame of reference;
automatically selecting, via the processing system, one or more candidate groups from the one or more series of MR images that were selected, wherein selection of the one or more candidate groups is based on predetermined rules;
automatically selecting, via the processing system, a respective reference image from each candidate group of the one or more candidate groups that were selected, wherein selection of each respective reference image is based on the predetermined rules; and
automatically displaying, via the processing system, the respective reference image selected for each candidate group of the one or more candidate groups that were selected, wherein each respective reference image is displayed in reference viewports on a user interface on a display based on the predetermined rules.

2. The computer-implemented method of claim 1, wherein the given MR image is a bolus observation plane image utilized to visualize and to track an incoming contrast bolus, each respective reference image that is selected and displayed is utilized to plan or adjust a bolus observation plane, and each respective reference image displays a respective cross-reference line indicating a location of the bolus observation plane image relative to the respective reference image.

3. The computer-implemented method of claim 2, wherein the predetermined rules comprise that each respective reference image is orthogonal to an orientation of the bolus observation plane image.

4. The computer-implemented method of claim 2, wherein the predetermined rules comprise that each respective reference image be selected from a series of MR images of the plurality of series of MR images is a non-localizer series of MR images when possible.

5. The computer-implemented method of claim 2, wherein the predetermined rules comprise that each respective reference image be selected that has its center closest to a center of the bolus observation plane image.

6. The computer-implemented method of claim 2, wherein the predetermined rules comprise displaying respective reference images of different reference orientations in the reference viewports, and, when respective reference images of different reference orientations are not available, displaying respective reference images having a same reference orientation.

7. The computer-implemented method of claim 2, wherein the predetermined rules comprise displaying, when no respective reference image is orthogonal to an orientation of the given MR image, respective reference images having a same orientation as the given MR image.

8. The computer-implemented method of claim 2, wherein the reference viewports comprises a first viewport and a second viewport on the user interface, and wherein the predetermined rules comprise displaying the respective reference images in the first viewport over the second viewport in a priority order of an axial image, a sagittal image, and a coronal image.

9. The computer-implemented method of claim 1, further comprising receiving, via the processing system, a selection from a user of a different reference image from a respective candidate group to display instead of displaying the respective reference image that was automatically selected from the respective candidate group.

10. The computer-implemented method of claim 1, further comprising receiving, via the processing system, a selection of a reference image from a user from a respective candidate group.

11. The computer-implemented method of claim 10, further comprising displaying, via the processing system, in one of the reference viewports either the reference image selected by the user or the respective reference image selected for each candidate group of the one or more candidate groups that were selected, wherein the user interface is configured to switch between displaying the reference image selected by the user or the respective reference image selected for each candidate group of the one or more candidate groups that were selected based on input from the user.

12. The computer-implemented method of claim 1, further comprising automatically displaying, via the processing system, in one of the reference viewports an initial reference image from a respective candidate group.

13. The computer-implemented method of claim 12, further comprising displaying, via the processing system, in one of the reference viewports the initial reference image and the respective reference image selected for each candidate group of the one or more candidate groups that were selected, wherein the user interface is configured to switch between displaying the initial reference image and the respective reference image selected for each candidate group of the one or more candidate groups that were selected based on input from the user.

14. The computer-implemented method of claim 13, further comprising receiving, via the processing system, a selection of a reference image from a user from a respective candidate group, and displaying, via the processing system, in one of the reference viewports one of the reference image, the initial reference image, and the respective reference image selected for each candidate group of the one or more candidate groups that were selected, and wherein the user interface is configured to switch between displaying the reference image, the initial reference image, and the respective reference image selected for each candidate group of the one or more candidate groups that were selected based on input from the user.

15. A system for selecting reference images for a magnetic resonance (MR) scan, comprising:
a memory encoding processor-executable routines; and
a processing system comprising one or more processors and configured to access the memory and to execute the processor-executable routines, wherein the processor-executable routines, when executed by the processing system, cause the processing system to:
for a given MR image acquired with an MR scanner and having an anatomical landmark and a frame of reference:
automatically select one or more series of MR images from a plurality of series of MR images previously acquired with the MR scanner and having the anatomical landmark and the frame of reference;
automatically select one or more candidate groups from the one or more series of MR images that were selected, wherein selection of the one or more candidate groups is based on predetermined rules;
automatically select a respective reference image from each candidate group of the one or more candidate groups that were selected, wherein selection of each respective reference image is based on the predetermined rules; and
automatically display the respective reference image selected for each candidate group of the one or more candidate groups that were selected, wherein each respective reference image is displayed in reference viewports on a user interface on a display based on the predetermined rules.

16. The system of claim 15, wherein the given MR image is a bolus observation plane image utilized to visualize and to track an incoming contrast bolus, each respective reference image that is selected and displayed is utilized to plan or adjust a bolus observation plane, and each respective reference image displays a respective cross-reference line indicating a location of the bolus observation plane image relative to the respective reference image.

17. The system of claim 16, wherein the predetermined rules comprise that each respective reference image is orthogonal to an orientation of the bolus observation plane image.

18. A non-transitory computer-readable medium, the computer-readable medium comprising processor-executable code that when executed by a processing system comprising one or more processors, causes the processing system to:
for a given magnetic resonance (MR) image acquired with an MR scanner for an MR scan and having an anatomical landmark and a frame of reference:
automatically select one or more series of MR images from a plurality of series of MR images previously acquired with the MR scanner and having the anatomical landmark and the frame of reference;
automatically select one or more candidate groups from the one or more series of MR images that were selected, wherein selection of the one or more candidate groups is based on predetermined rules;
automatically select a respective reference image from each candidate group of the one or more candidate groups that were selected, wherein selection of each respective reference image is based on the predetermined rules; and
automatically display the respective reference image selected for each candidate group of the one or more candidate groups that were selected, wherein each respective reference image is displayed in reference viewports on a user interface on a display based on the predetermined rules.

19. The non-transitory computer-readable medium of claim 18, wherein the given MR image is a bolus observation plane image utilized to visualize and to track an incoming contrast bolus, each respective reference image that is selected and displayed is utilized to plan or adjust a bolus observation plane, and each respective reference image displays a respective cross-reference line indicating a location of the bolus observation plane image relative to the respective reference image.

20. The non-transitory computer-readable medium of claim 19, wherein the predetermined rules comprise that each respective reference image is orthogonal to an orientation of the bolus observation plane image.
